(19) United States
(12) Patent Application Publication
(10) Pub. No.: US 2019/0133540 A1
(43) Pub. Date: May 9, 2019

(54) EPICARDIAL ADIPOSE TISSUE INFLAMMATORY INDEX PREDICTION OF LESION-SPECIFIC ISCHEMIA AS ASSESSED BY FRACTIONAL FLOW RESERVE

(71) Applicant: Loyola University of Chicago, Maywood, IL (US)

(72) Inventor: Mark Rabbat, Oak Brook Terrace, IL (US)

(21) Appl. No.: 16/180,113

(22) Filed: Nov. 5, 2018

Related U.S. Application Data
(60) Provisional application No. 62/582,071, filed on Nov. 6, 2017.

(51) Int. Cl. A61B 6/00; A61B 6/03

(52) U.S. Cl. CPC A61B 6/504; A61B 5/02007; A61B 6/032; A61B 6/5217

(57) ABSTRACT

Medical procedures for predicting lesion-specific ischemia using a technique that incorporates pericoronary epicardial adipose tissue (EAT) inflammation assessed by an inflammatory index that can be quantified by computed tomography (CT). Such a procedure includes using a CT technique to measure a total volume of EAT, using a CT technique to determine density of pericoronary EAT, calculating a pericoronary EAT CT density gradient (PDG) as a difference in density between the pericoronary EAT and EAT remote from a coronary artery of the individual, determining an inflammatory index value by multiplying PDG by the total volume of the EAT, and predicting vessel-specific ischemia of the artery based on the inflammatory index value.

### CROSS REFERENCE TO RELATED APPLICATIONS

This application claims the benefit of U.S. Provisional Application No. 62/582,071, filed Nov. 6, 2017, the contents of which are incorporated herein by reference.

### BACKGROUND OF THE INVENTION

The present invention generally relates to techniques for assessing artery stenosis (stenotic lesions) to determine the extent to which stenosis impedes blood flow in a vessel.

Fractional flow reserve (FFR) is a known technique used to measure pressure differences across a coronary artery stenosis (stenotic lesion) to determine the extent to which the stenosis impedes blood flow, for example, to the heart muscle (myocardial ischemia). FFR is calculated as the ratio of maximum blood flow distal to a stenosis to normal maximum flow in the same vessel, calculated using the following equation:

FFR=p_{d}/pₐ

where p_{d} is the blood pressure distal to the stenosis and pₐ is the pressure proximal to the stenosis. FFR has been determined invasively using catheterization techniques to place a transducer in the vessel being assessed. Computation of FFR has also been derived noninvasively from coronary computed tomography (CT) angiography (CTA).

Epicardial adipose tissue (EAT) is a metabolically active organ which has been associated with the presence and severity of coronary artery disease. The relationship between pericoronary EAT inflammation and hemodynamically significant coronary artery disease assessed by FFR has not been studied. The presence of inflammation in EAT results in a higher density (in Hounsfield Unit, HU) in CT images.

### BRIEF DESCRIPTION OF THE INVENTION

The present invention provides a medical procedure for predicting lesion-specific ischemia using a technique that incorporates inflammation of pericoronary epicardial adipose tissue (EAT) (defined as adipose tissue within the pericardium of an individual) assessed by an inflammatory index that can be quantified by computed tomography (CT).

According to one aspect of the invention, the procedure includes using a CT technique to measure a total volume of EAT, using a CT technique to determine density of pericoronary EAT, calculating a pericoronary EAT CT density gradient (PDG) as a difference in density between the pericoronary EAT and EAT remote from a coronary artery of the individual, determining an EATi value by multiplying PDG by the total volume of the EAT, and predicting vessel-specific ischemia of the artery based on the EATi value.

Technical aspects of the procedure described above include the ability to employ noninvasive CT-based quantification of EAT inflammation to help differentiate functionally significant from non-functional CT coronary stenosis.

Other aspects and advantages of this invention will be further appreciated from the following detailed description.

### BRIEF DESCRIPTION OF THE DRAWINGS

FIGS. 1A and 1B depict representative examples of two different human patients from a study that demonstrated invasive FFR and FFR_{CT} values and corresponding EATi, where EATi is a numerical value calculated as pericoronary EAT CT density gradient multiplied by total EAT volume, FFR is the fractional flow reserve determined invasively by catheterization, and FFR_{CT} is the fractional flow reserve determined by CT. FIG. 1A represents a vessel for which functionally significant invasive FFR and FFR_{CT} values of 0.61 and 0.68, respectively, were determined, along with an EATi of −1387. FIG. 1B represents a vessel for which functionally insignificant invasive FFR and FFR_{CT} values of 0.95 and 0.92, respectively, were determined, along with EATi of 225. The results indicated a significant correlation between EATi and FFR values.

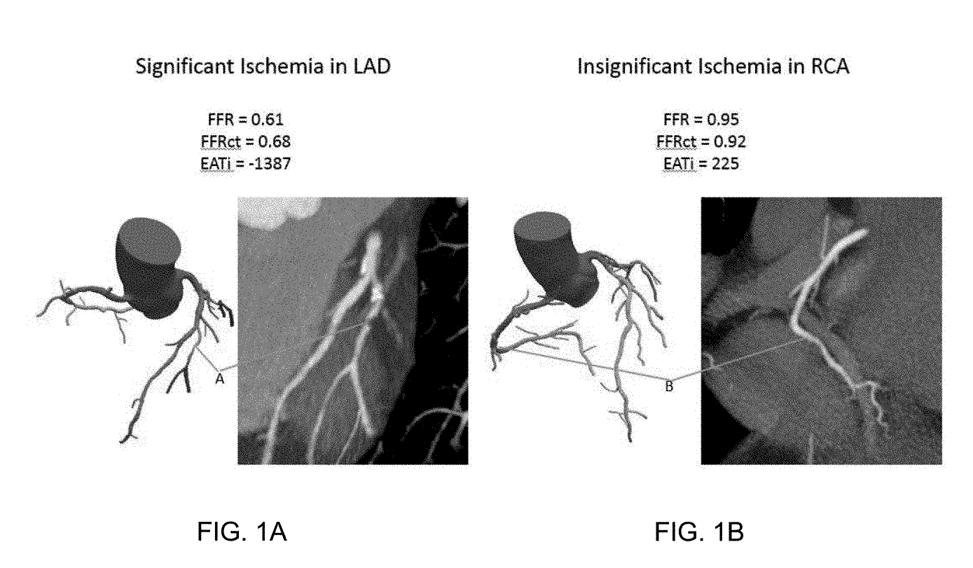

### DETAILED DESCRIPTION OF THE INVENTION

Current computation of fractional flow reserve (FFR) derived from coronary computed tomography angiography datasets (FFR_{CT}) using equipment and methodologies of HeartFlow, Inc. (and possibly other vendors, such as Toshiba, Siemens, General Electric, Philips, etc.) does not incorporate epicardial adipose tissue (EAT) measurements. During investigations leading to the present invention, it was hypothesized that incorporating EAT variables such as adipose composition derived from computed tomography (CT) may be capable of predicting plaque formation, intimal thickening, myocardial fibrosis, valvular stenosis, diastolic dysfunction, FFR, adverse plaque characteristics, acute coronary syndrome, cardiac and cardiovascular death, and all-cause mortality, impact plaque stress, and improve the diagnostic performance of FFR derived from coronary computed tomography angiography. Furthermore, it was hypothesized that parameters of EAT derived from coronary CT datasets might help differentiate functionally significant from non-functional CT coronary stenosis. EAT parameters may also help in accurately determining rest and hyperemic coronary flow and pressure and impact the mathematical model of coronary physiology to derive boundary conditions such as microcirculatory/microvascular resistance. As there is heterogeneity in the composition of EAT within an individual and coronary artery territories, the unique resistance of each individual coronary outlet will vary. Furthermore, EAT parameters may positively influence the assumptions in the physiological models and simulation of maximal hyperemia when analyzing FFR derived from coronary computed tomography angiography, specifically in patients with microvascular disease to improve the current overestimation of the degree of vasodilation in the current algorithm. Hence, it was further hypothesized that incorporating EAT parameters may be capable of improving the diagnostic performance of FFR derived from coronary computed tomography angiography, FFR_{CT} particularly as determined with methodologies offered by HeartFlow, Inc., and possibly other coronary CTA-derived FFR algorithms that may be or have been offered by other vendors (Toshiba, Siemens, General Electric, Philips).

To predict lesion-specific ischemia, an investigation leading to the present invention developed what is referred to herein as an EAT inflammatory index (EATi), which was quantified by CT to assess pericoronary EAT inflammation. In the investigation, thirty-two human patients from different trials were included. Fifty-one vessels of these patients having measured invasive FFR values were studied. For purposes of the research, an FFR value of 0.80 was considered to be diagnostic of lesion-specific ischemia. Total EAT volume (mL), defined as adipose tissue within the pericardium, was measured using contrast-enhanced CT. Total EAT was quantified using axial slices from the right pulmonary artery to the diaphragm with a threshold of −30 to −190 HU on a commercially available workstation. After thresholding for adipose, circular halo's 1 mm external to the vessel luminal boundary determined the pericoronary EAT density. The density of remote EAT was measured with a halo 5 mm in diameter distant from the coronary arteries. Pericoronary EAT CT density gradient (PDG) was defined as the difference in CT density between pericoronary EAT and EAT remote from the coronary artery. EATi was defined as PDG×total EAT volume.

Ischemic arteries as determined by invasive FFR (catheterization) were associated with a sixfold difference in EATi (−1082±1893 versus 213±1751, p=0.015) compared to non-ischemic arteries. Logistic regression demonstrated EATi to be a significant predictor of vessel-specific ischemia (p=0.028). There was no difference in EATi between stenotic and non-stenotic vessels as determined by CT (−538±2353 versus −110±1358, p=0.4335). Among patients with great than 50% CT stenosis, EATi was significantly different in ischemic lesions than in non-functional stenosis (−1589±2121 versus 799±1981, p=0.009), thus aiding in the differentiation between true and false positive CT. Using a threshold EATi value of 2000, 27% of CT stenosis false positives were correctly reclassified to true negatives without misclassifying any true positives.

The investigation was concluded to have demonstrated that a CT-based quantification of EAT inflammation can be associated with lesion-specific ischemia to help differentiate functionally significant from non-functional CT coronary stenosis in a human.

While the invention has been described in terms of a specific investigation, it should be apparent that alternatives could be adopted by one skilled in the art. Accordingly, it should be understood that the invention is not necessarily limited to any embodiment described herein. It should also be understood that the phraseology and terminology employed above are for the purpose of describing the disclosed investigation, and do not necessarily serve as limitations to the scope of the invention. Therefore, the scope of the invention is to be limited only by the following claims.

1. A medical procedure for assessing artery stenosis, the procedure comprising:
using a computed tomography (CT) technique to measure a total volume of epicardial adipose tissue (EAT) defined as adipose tissue within the pericardium of an individual;
using a CT technique to determine density of pericoronary EAT of the individual;
calculating a pericoronary EAT CT density gradient (PDG) as a difference in density between the pericoronary EAT and EAT remote from a coronary artery of the individual;
determining an EATi value by multiplying PDG×the total volume of the EAT; and
predicting vessel-specific ischemia of the artery based on the EATi value.

2. The medical procedure of claim 1, further comprising using a threshold EATi value to classify stenosis true positives and true negatives.

3. The medical procedure of claim 1, wherein the procedure is performed on a human to differentiate functionally significant from non-functional CT coronary stenosis in the human.
